(19) United States
(12) Patent Application Publication
(10) Pub. No.: US 2024/0058410 A1
(43) Pub. Date: Feb. 22, 2024

(54) METHOD FOR PREPARING A FEEDING INSTANT ASTRAGALUS POLYSACCHARIDE POWDER AND APPLICATION THEREOF

(71) Applicant: ZHEJIANG UNIVERSITY OF SCIENCE AND TECHNOLOGY, Zhejiang (CN)

(72) Inventors: Dehui DAI, Zhejiang (CN); Weilian HU, Zhejiang (CN); Guicai CHEN, Zhejiang (CN)

(73) Assignee: ZHEJIANG UNIVERSITY OF SCIENCE AND TECHNOLOGY, Zhejiang (CN)

(21) Appl. No.: 18/344,828

(22) Filed: Jun. 29, 2023

(30) Foreign Application Priority Data
Aug. 18, 2022 (CN) 202210990031.0

(51) Int. Cl. A61K 36/481; A23K 10/30; A23K 20/163; A61K 9/14; B01D 61/14; B01D 69/02; B01D 69/08

(52) U.S. Cl. CPC A61K 36/481; A23K 10/30; A23K 20/163; A61K 9/14; B01D 61/145; B01D 69/02; B01D 69/08; A61K 2236/331; A61K 2236/333; A61K 2236/37; A61K 2236/39; A61K 2236/53; B01D 2325/0283

(57) ABSTRACT

The present application is related to a method for preparing a feeding instant Astragalus polysaccharide powder and an application thereof. The method includes the following steps: adding deionized water at 65° C. to a dreg of Astragalus membranaceus by water extraction and alcohol precipitation, controlling an alcohol volume fraction at 20%, stirring fully to dissolve the dreg, and after a dreg solution cools to room temperature, removing insoluble thermo-sensitive macromolecular proteins, polysaccharides and solid residues by centrifugation at 3500 revolutions per minute to obtain a clear supernatant. Ultrafiltration treatment is performed by hollow fiber membranes on the supernatant obtained after the centrifugation, and 10-100 kDa polysaccharide components are collected. A low-molecular-weight instant Astragalus polysaccharide powder with high immunocompetence is obtained through an interlayer cold air cooling spray drying process, and the content of active polysaccharide is more than 87%.

### CROSS-REFERENCE TO RELATED APPLICATION

This application claims the priority benefit of China application no. 202210990031.0, filed on Aug. 18, 2022. The entirety of each of the above-mentioned patent application is hereby incorporated by reference herein and made a part of this specification.

### BACKGROUND OF THE INVENTION

### 1. Technical Field

The present invention belongs to the field of functional feed additives for livestock and poultry breeding, and specifically relates to a method of extracting highly active polysaccharide components using a dreg of Astragalus membranaceus by water extraction and alcohol precipitation in a traditional Chinese medicine factory, and preparing an instant Astragalus polysaccharide immune-enhancing powder, and an application of the powder.

### 2. Background Art

Astragalus membranaceus, the root of Astragalus membranaceus (Fisch.) Bge. and Astragalus membranaceus(Fisch.) Bge. var. mongholicus (Bge.) Hsiao in Leguminosae, is a widely used Chinese herbal medicine in China and the most commonly used qi-tonifying drug in clinical practice, with a history of over 2000 years. Modern pharmaceutical research has shown that Astragalus membranaceus contains various active ingredients, including Astragalus saponins, Astragalus flavonoids, Astragalus polysaccharides, and other main functional components. It plays an important role in immune regulation, anti-tumor and anti-virus aspects, diabetes, delaying senescence and so on. Astragalus polysaccharides, one of the important active ingredients in Astragalus membranaceus, have the characteristics of safety, high efficiency, no residue and the like. Moreover, they have various pharmacological effects such as enhancing animal growth performance, improving immune performance, and increasing antioxidant capacity. Previous studies on the application or pharmacological activity of Astragalus polysaccharides have mainly focused on total polysaccharides. Industrial products such as Astragalus polysaccharide oral liquid and Astragalus polysaccharide feed additives all use total polysaccharides. The preparation method of total polysaccharides is relatively extensive, and even if the same extraction method is used, the total polysaccharide components obtained may differ due to specific production raw materials or parameter differences between batches, which inevitably leads to the instability of the pharmacological activity of crude polysaccharides. Therefore, segmenting Astragalus polysaccharides according to the size of molecular weight to obtain relatively uniform components is of great significance for improving the pharmacological activity and stability of Astragalus polysaccharides. Astragalus polysaccharides are mainly divided into large-molecule components and small-molecule components. Polysaccharide components with average molecular weight of 10-100 kDa are the main active components in Astragalus polysaccharides. However, due to high thermal sensitivity, the polysaccharide components are difficult to prepare using ordinary drying methods. At the same time, the resources of Astragalus membranaceus in China are limited, and directly using the traditional Chinese medicine Astragalus membranaceus to extract Astragalus polysaccharides is costly and difficult to widely apply in the production of poultry, pigs, ruminants, and aquatic animals.

At present, the related products of Astragalus polysaccharides are mainly concentrated on total Astragalus polysaccharides, resulting in poor pharmacological activity and stability of the products. Low-molecular-weight Astragalus polysaccharide components of 10-100 kDa are the most biologically active part of Astragalus polysaccharides. But they have extremely high thermal sensitivity, and are prone to sticking, foaming, and browning under high-temperature conditions. Therefore, a small number of Astragalus polysaccharide samples are usually processed by a freeze-drying method, which has higher production costs and smaller processing capacity. The spray-drying method is a better way to deal with a large number of Astragalus polysaccharides. However, due to high thermal sensitivity and wall adhesion of Astragalus polysaccharides, it is difficult to obtain dry powder. More than 30% maltodextrin is usually added for mixing treatment, but it is difficult to obtain a pure product of Astragalus polysaccharides, and some manufacturers use the defects in the current detection methods of polysaccharides to mix a large amount of maltodextrin with Astragalus polysaccharides.

The dreg of Astragalus membranaceus by water extraction and alcohol precipitation is the residual waste after water-soluble small-molecule components such as astragaloside IV are extracted from Astragalus membranaceus by traditional Chinese medicine enterprises using the water extraction and alcohol precipitation method. Due to the semi-solid paste-like nature and high viscosity of the dreg, it is not conducive to transportation and stacking, belongs to one of the most difficult categories of waste for environmental protection companies to handle, and meanwhile, is also the most expensive part of the dreg treatment process. However, the semi-solid waste residue of Astragalus membranaceus by water extraction and alcohol precipitation contains a large number of bioactive macromolecules such as polysaccharides. After analysis, the polysaccharide content accounts for over 70% of the dry matter. The use of highly active Astragalus polysaccharide components extracted from the dreg of Astragalus membranaceus by water extraction and alcohol precipitation can reduce the environmental pressure of traditional Chinese medicine enterprises, while also reducing the production cost of Astragalus polysaccharides, which is conducive to the widespread application of Astragalus polysaccharides in the livestock and poultry breeding industry. The instant Astragalus polysaccharide immune-enhancing powder prepared by the present invention not only has low costs and fast product dissolution, but also has high food safety and good immune-enhancing effects on the body through animal experiments.

### SUMMARY OF THE INVENTION

The purpose of the present invention is to provide a method for preparing a feeding instant Astragalus polysaccharide powder and an application thereof, in response to the problems existing in the prior art. The prior art mainly focuses on the related products of Astragalus polysaccharides, resulting in poor pharmacological activity and stability of the products, which is to be solved by the present invention.

The present invention is specifically implemented through the following technical solutions:

A method for preparing a feeding instant Astragalus polysaccharide powder, including the following steps:
1) adding deionized water at 65° C. to a dreg of Astragalus membranaceus by water extraction and alcohol precipitation, and controlling an alcohol volume fraction at 20%;
2) stirring fully to dissolve the dreg of Astragalus membranaceus by water extraction and alcohol precipitation, and after a dreg solution cools to room temperature, removing insoluble thermo-sensitive macromolecular proteins, polysaccharides and solid residues by centrifugation at 3500 revolutions per minute to obtain a clear supernatant; and
3) adding twice mass of deionized water to the supernatant obtained after the centrifugation, stirring fully, and then performing ultrafiltration treatment by a hollow fiber membrane with a pore size of 100 KDa, and collecting components below 100 kDa. Then 10-100 kDa polysaccharide components are collected by ultrafiltration treatment with a 10 kDa hollow fiber membrane.

Preferably, the polysaccharide component with molecular weight of 10-100 kDa is taken after two ultrafiltration treatments in the step 3), concentrated under vacuum and low temperature to 40% of the solid content of the component, and dried by spray to obtain a highly active low-molecular-weight Astragalus polysaccharide powder.

Preferably, conditions of the spray drying are as follows: an atomizer frequency is 70 Hz, inlet air temperature is controlled at 140° C., outlet air temperature is controlled below 60° C., a drying tower interlayer is cooled by cold air, and a bottom of a drying tower is vibrated by a vibrating hammer every 5 minutes to prevent a wall and the bottom of the drying tower from powder deposition and liquefaction foaming.

The feeding instant Astragalus polysaccharide powder prepared by any of the above preparation methods is applied in a feed additive.

The dreg of Astragalus membranaceus by water extraction and alcohol precipitation in the present invention is a semi-solid residue obtained by extracting traditional Chinese medicine Astragalus membranaceus pieces with hot water, removing a solid residue, and adding 3-4 times volume of anhydrous ethanol.

The preparation method of the present invention not only can effectively utilize the waste dreg of Astragalus membranaceus by water extraction and alcohol precipitation to reduce environmental pollution, but also can prepare an instant Astragalus polysaccharide powder with high immunocompetence, which can have a powder yield of spray drying reaching 90%, and has an active polysaccharide content more than 87% and a drying weight loss less than 6%. The instant Astragalus polysaccharide powder with high immunocompetence obtained by the present invention is low in production costs and high in biological activity, can be used as a feed additive to be applied to livestock and poultry breeding, replaces or reduces use of antibiotics, and has relatively high economic value.

### BRIEF DESCRIPTION OF THE DRAWINGS

FIG. 1 shows a dreg of Astragalus membranaceus by water extraction and alcohol precipitation;

FIG. 2 shows the grading ratio of ethanol precipitation in a dreg of Astragalus membranaceus by water extraction and alcohol precipitation;

FIG. 3 shows molecular weight distribution in a 20% alcohol precipitate of a dreg of Astragalus membranaceus by water extraction and alcohol precipitation;

FIG. 4 shows an instant highly active low-molecular-weight Astragalus polysaccharide powder;

FIG. 5 shows changes in body weight of mice fed for 30 days;

FIG. 6 shows organ coefficients of mice fed for 30 days;

FIG. 7 shows an effect of Astragalus polysaccharide powder on body weight of mice (compared with the control group, *, P<0.05, * *, P<0.01; compared with the model group, #, P<0.05, ##, P<0.01; unmarked data in the same column indicate no significant difference (P>0.05));

FIG. 8 shows an effect of Astragalus polysaccharide powder on coefficients of immune organs and organs in mice (compared with the control group, *, P<0.05, * *, P<0.01; compared with the model group, #, P<0.05, ##, P<0.01; unmarked data in the same column indicate no significant difference (P>0.05));

FIG. 9 shows an effect of Astragalus polysaccharide powder on y-interferon in peripheral blood (compared with the control group, *, P<0.05, * *, P<0.01; compared with the model group, #, P<0.05, ##, P<0.01; unmarked data in the same column indicate no significant difference (P>0.05));

FIG. 10 shows an effect of Astragalus polysaccharide powder on IgG in peripheral blood (compared with the control group, *, P<0.05, * *, P<0.01; compared with the model group, #, P<0.05, ##, P<0.01; unmarked data in the same column indicate no significant difference (P>0.05)); and

FIG. 11 shows an effect of Astragalus polysaccharide powder on T lymphocyte subpopulations in peripheral blood of mice (compared with the control group, *, P<0.05, * *, P<0.01; compared with the model group, #, P<0.05, ##, P<0.01; unmarked data in the same column indicate no significant difference (P>0.05)).

### DETAILED DESCRIPTION OF THE EMBODIMENTS

The following provides a further detailed description of the present invention in conjunction with specific embodiments, in order to better understand the technical solutions.

### Embodiment 1

Deionized water that had been heated to 65° C. was added to 50 kilograms of a dreg of Astragalus membranaceus by water extraction and alcohol precipitation from a production enterprise of Astragalus membranaceus injection, the amount of water added was controlled with a precision alcohol meter to control a final alcohol volume fraction at 20%, and stirring lasted for 20 minutes until the dreg of Astragalus membranaceus by water extraction and alcohol precipitation is evenly dissolved. After a dreg solution cooled to room temperature, insoluble residues were removed by centrifugation at 3500 revolutions per minute to obtain a clear supernatant; twice mass of deionized water was added to the supernatant and stirred evenly, and then ultrafiltration was performed by an ultrafiltration membrane with the molecular weight cut-off of 100 KDa, and liquid components below 100 kDa were collected. Then 10-100 kDa polysaccharide components were collected by ultrafiltration treatment with a 10 kDa hollow fiber membrane. Further, through evaporation and concentration under vacuum and low temperature, a concentrated trapped solution with a solid content of 40% was obtained. A spray dryer was used for drying, an atomizer frequency was 70 Hz, inlet air temperature was controlled at 140° C., outlet air temperature was selected at 60° C., a feeding speed was 15 kilograms per hour, a drying tower interlayer was cooled by cold air below 20° C., a bottom of a drying tower was hammered with a hammer every 5 minutes to prevent powder deposition on a wall of the drying tower and liquefaction foaming of high-temperature active polysaccharides, and a dried powder was collected in a workshop equipped with dehumidification equipment to obtain highly a bioactive Astragalus polysaccharide powder. Its appearance was light yellow, easy to absorb moisture, and highly soluble in water, and had no bad odor. A powder yield of spray drying was about 90%, a content of crude polysaccharide was 87%, a drying weight loss was 6%, solvent residues were not detected, a content of heavy metals (arsenic+lead+cadmium+mercury) was <10.0 ppm, and pathogenic bacteria such as coliform bacteria and salmonella were not detected. The highly active low-molecular-weight Astragalus polysaccharide powder has extremely high hygroscopicity, and should be sealed and stored after collection.

### Experimental Example 1: Composition Analysis of a Dreg of Astragalus membranaceus by Water Extraction and Alcohol Precipitation and Selection of a Spray Drying Process

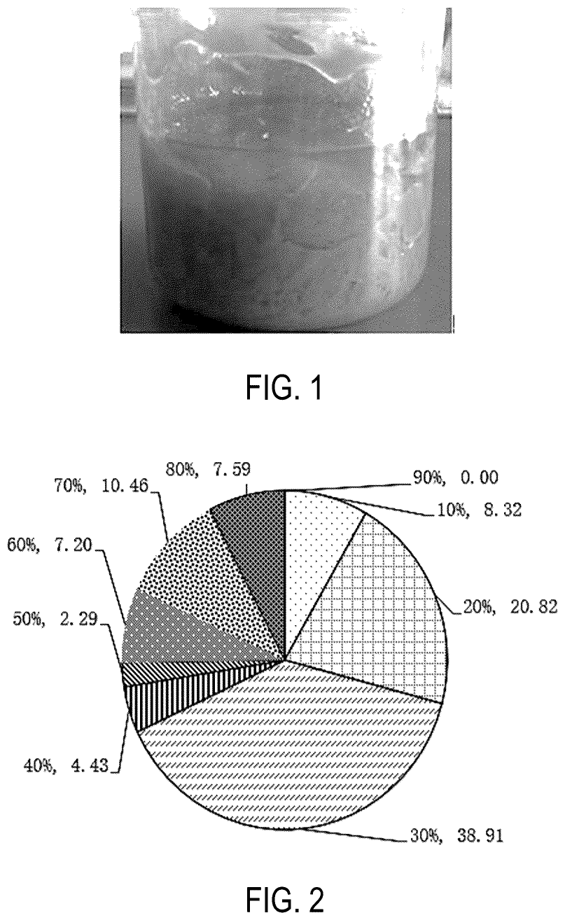

Analysis of main components of the dreg of Astragalus membranaceus by water extraction and alcohol precipitation: the dreg of Astragalus membranaceus by water extraction and alcohol precipitation is the residual waste after water-soluble small-molecule components such as astragaloside IV are extracted from Astragalus membranaceus using the water extraction and alcohol precipitation method. Due to the semi-solid paste-like nature and high viscosity of the dreg, as shown in FIG. 1, its main components are water-soluble active polysaccharides and proteins, as shown in Table 1. Astragalus polysaccharides can be used as immune enhancers or modulators, and have antiviral, anti-tumor, anti-aging, anti-radiation, anti-stress, and antioxidant effects and the like. Therefore, it has extremely high health and nutritional value and can be developed into animal health products or drugs.

TABLE 1
Composition analysis of a dreg of Astragalus membranaceus
by water extraction and alcohol precipitation

| | Dry matter/% | | | | |
|---|---|---|---|---|---|
| Drying | Crude | Crude | Free | Reducing | Ash |
| loss/% | polysaccharide | protein | protein | sugar | content |
| 36.15 | 72.32 | 13.14 | 1.15 | 2.08 | 2.62 |

Grading of polysaccharide alcohol precipitation in the dreg of Astragalus membranaceus by water extraction and alcohol precipitation: the dreg of Astragalus membranaceus by water extraction and alcohol precipitation was dissolved in water with an ethanol concentration below 1%, a supernatant was collected by centrifugation, ethanol was added to the supernatant to a concentration of 10%, a precipitate was collected, then the ethanol concentration of the supernatant was adjusted to 20%, a precipitate was collected, the ethanol concentration of the supernatant was adjusted to 30% and so on, until the ethanol concentration was 90%. The alcohol precipitate was dried by freezing, and the proportion of polysaccharides by alcohol precipitation in each part was analyzed. FIG. 2 showed that polysaccharides under an alcohol concentration of 30% accounted for the largest proportion, followed by 20% and 10%, while alcohol precipitates were fewer under 50% and 40% concentrations, indicating that the distribution of polysaccharides in different alcohol precipitated parts of Astragalus polysaccharides is very uneven. Polysaccharides with different molecular weight have different solubility in different concentrations of ethanol. Large-molecule polysaccharides are prone to precipitation in lower concentrations of ethanol solutions, while small-molecule polysaccharides are prone to dissolution in higher concentrations of ethanol.

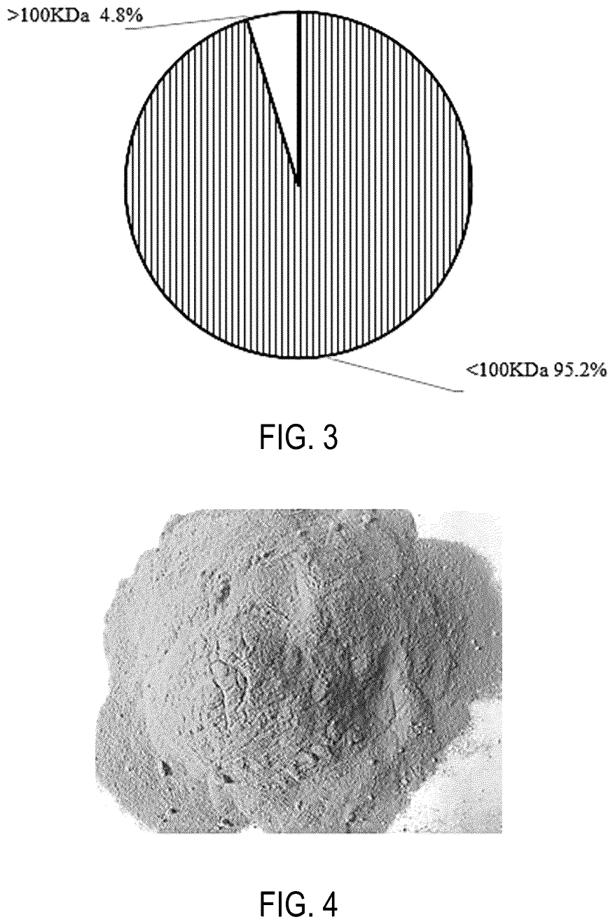

Molecular weight distribution in a 20% alcohol precipitate of a dreg of Astragalus membranaceus by water extraction and alcohol precipitation: the dreg of Astragalus membranaceus by water extraction and alcohol precipitation was dissolved in deionized water until the alcohol content was 20%. Centrifugation was performed to collect precipitates, the precipitates were then added with deionized water for complete dissolution, a supernatant was collected by centrifugation, the supernatant is then passed through a hollow fiber membrane with a molecular weight of 100 KDa to collect a trapped solution, and the polysaccharide content in the supernatant and the trapped solution was measured separately, and the ratio between the two was calculated. FIG. 3 showed that in the 20% alcohol precipitate of a dreg of Astragalus membranaceus by water extraction and alcohol precipitation, components below 100 KDa accounted for less than 5%. If the dreg of Astragalus membranaceus by water extraction and alcohol precipitation was dissolved in deionized water until the alcohol content was 20%, and the precipitates were removed by centrifugation, the loss of target components (10-100 KDa molecular weight) was minimal, but the efficiency of subsequent ultrafiltration can be effectively improved.

Influence of air inlet temperature on spray drying of Astragalus polysaccharides: the 10-100 kDa components of Astragalus polysaccharides were adjusted to about 40% of solid contents. At this concentration, it had good fluidity and high solid contents, which was conducive to improving the efficiency of spray drying. Air outlet temperature was controlled below 60° C., an atomizer frequency was 70 Hz, and four temperature gradients including 140, 160, 180, 200° C. were selected. The results are shown in Table 2.

TABLE 2
Effects of air inlet temperature on spray
drying of astragalus polysaccharides

| Inlet air | | | |
|---|---|---|---|
| temperature/ | Powder | Water | |
| ° C. | yield/% | content/% | Sensory effects |
| 140 | 89.52 | 6.0 | Slight wall sticking, good spray |
| | | | effect, fine and even powder |
| | | | particles, loose, slightly |
| | | | caking, and light yellow |
| 160 | 70.17 | 5.3 | Foaming and wall sticking, general |
| | | | spray effect, fine and even powder |
| | | | particles, loose, caking, and light |
| | | | brown yellow |
| 180 | 48.18 | 4.5 | Severe foaming and wall sticking, |
| | | | good spray effect, caking, and |
| | | | brown yellow |
| 200 | 30.71 | 3.8 | Severe foaming and wall sticking, |
| | | | difficult to obtain dry powder, large |
| | | | caking, and brown yellow |

Table 2 showed that when the inlet air temperature was 140° C., it was equipped with interlayer cold air. Astragalus polysaccharides slightly sticked to the wall, and spray drying was effective. The obtained Astragalus polysaccharides had a light color, as shown in FIG. 4. They were light yellow in color, free from caking, easy to absorb moisture, and highly soluble in water.

### Experimental Example 2: 30-Day Feeding Experiment on Mice Using a Feeding Instant Astragalus Polysaccharide Immune-Enhancing Powder

Experiment method: 80 clean-grade ICR mice weighing 18-24 g were randomly divided into 4 groups, with 20 mice in each group, half male and half female. Experimental groups were divided into three dose groups: high, medium, and low, with doses of 1.0, 2.0, and 4.0 mg kg⁻¹ d⁻¹, respectively. Another control group was set up, and an equal dose of distilled water was given by gavage every day. General observation: during the experiment, the general state, physical signs, food intake, water consumption, and fecal status of mice were observed every day. The body weight was measured every 7 days, the dosage was adjusted according to changes in the body weight, and mice were continuously observed for 30 days. Measurement of hematology indicators: after 30 days of drug administration and 12 hours of fasting and water deprivation, eyeballs were removed and blood was taken to examine hematology indicators. Hematology examination items included: white blood cell count (WBC), percentage of neutrophils (NE %), percentage of lymphocytes (LY %), percentage of monocytes (MO %), percentage of eosinophils (EO %), basophils (BA %), red blood cell count (RBC), hemoglobin concentration (Hb), hematocrit (HCT %), mean corpuscular volume (MCV), mean corpuscular hemoglobin content (MCH), mean corpuscular hemoglobin concentration (MCHC), red blood cell distribution width (RDW %), platelet count (PLT), and mean platelet volume (MPV). Measurement of blood biochemical indicators: After 30 days of drug administration and 12 hours of fasting and water deprivation, eyeballs were removed and blood was taken to examine blood biochemical indicators. The blood biochemical indicators included: glutamic pyruvic transaminase (ALT/GPT), glutamic oxalacetic transaminase (AST/GOT), total protein (TP), albumin (ALB), blood urea nitrogen (BUN), creatinine (CRE), total cholesterol (CHO), triglyceride (TG), and glucose (GLU). Observation of gross morphological changes: immediately after blood collection, organs such as the liver, kidney, spleen, stomach, testicles, or ovaries of the animals were dissected and weighed, and organ weight coefficients were calculated based on the body weight. Moreover, the morphological changes were observed with the naked eye.

Experiment Results:

General toxic reactions in mice: during the 30-day experimental period, the mice in the high, medium, and low dose administration groups and the control group showed normal activity, active behavior, glossy fur, normal food and water consumption, no abnormal changes in defecation and urination, and no deaths.

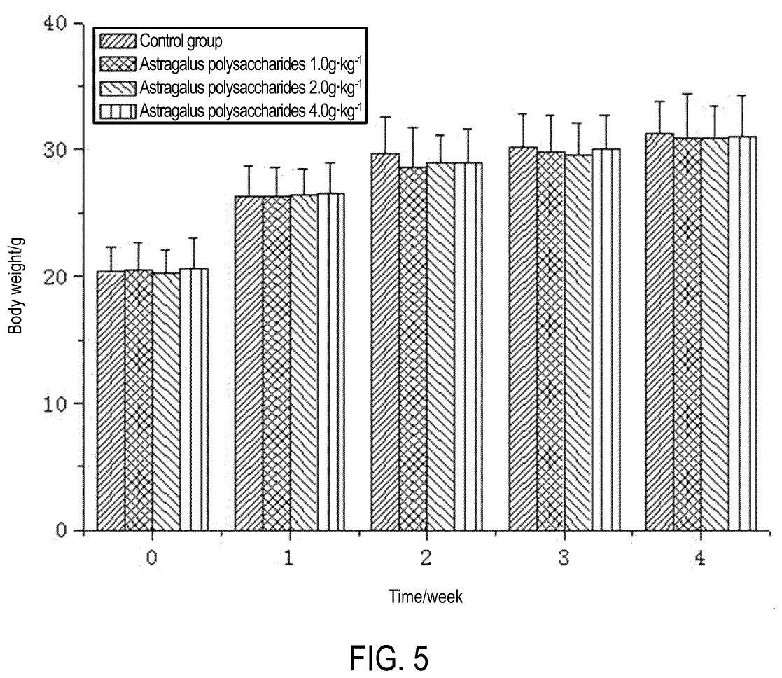

Body weight changes of mice: the changes in body weight after 30 days of feeding are shown in FIG. 5. FIG. 5 showed that the weight growth of the high, medium, and low dose administration groups was normal, and compared with the control group, no significant difference was shown in the body weight (P>0.05).

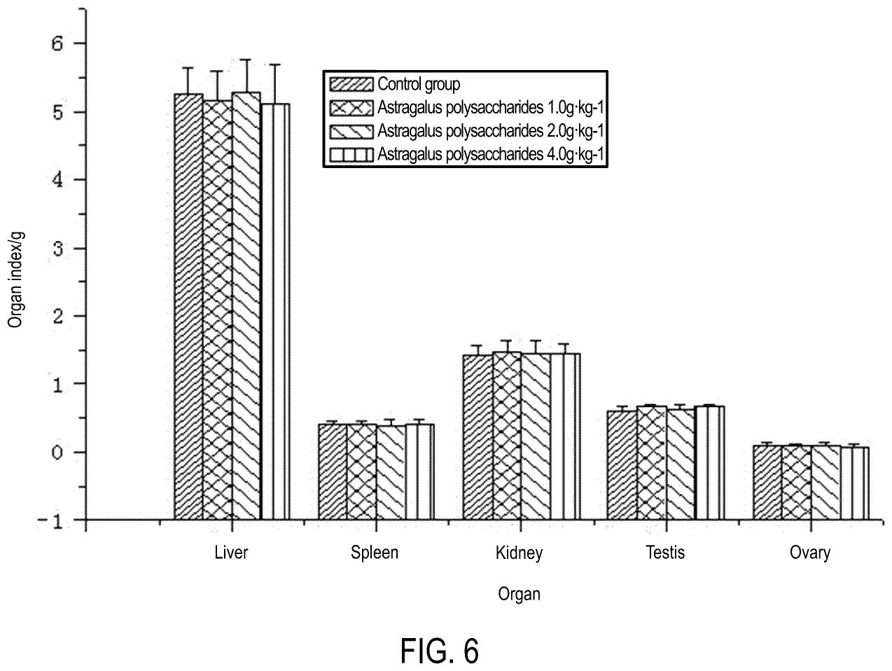

Organ coefficients of mice: after 30 days of feeding, the organ coefficients of mice are shown in FIG. 6. FIG. 6 showed that after 30 days of administration of the Astragalus polysaccharide powder, there was no significant difference in organ coefficients between each dose group and the control group (P>0.05).

Blood routine indicators: The results of the effects of the Astragalus polysaccharide powder on blood routine indicators in mice are shown in Table 3.

TABLE 3
Effects of the astragalus polysaccharide powder on blood routine in mice (x̅ ± s)

| | | Measurement indicators | | | | |
|---|---|---|---|---|---|---|
| Groups | | WBC (K · μl⁻¹) | RBC(M · μl⁻¹) | HB(g · dl⁻¹) | HCT(%) | MCV(fL) |
| Control group | | 8.01 ± 2.45 | 10.11 ± 2.54 | 14.32 ± 3.93 | 55.06 ± 12.58 | 54.66 ± 7.69 |
| Sample | 1.0 | 7.01 ± 2.86 | 10.22 ± 1.45 | 13.68 ± 2.51 | 55.61 ± 10.68 | 52.85 ± 6.93 |
| group | 2.0 | 7.32 ± 1.58 | 10.03 ± 2.55 | 14.26 ± 2.33 | 54.29 ± 14.59 | 56.15 ± 10.19 |
| (g · kg⁻¹) | 4.0 | 7.26 ± 3.08 | 10.15 ± 2.38 | 13.15 ± 3.23 | 51.69 ± 18.95 | 54.65 ± 9.76 |

| | | Measurement indicators | | | | |
|---|---|---|---|---|---|---|
| Groups | | MPV(fL) | MCH(Pg) | NE(%) | LY(%) | EO(%) |
| Control group | | 5.07 ± 0.33 | 13.96 ± 1.15 | 24.25 ± 9.25 | 65.26 ± 9.22 | 1.21 ± 2.35 |
| Sample | 1.0 | 4.95 ± 0.56 | 14.12 ± 0.95 | 25.65 ± 11.65 | 62.26 ± 10.80 | 0.66 ± 0.78 |
| group | 2.0 | 5.12 ± 0.35 | 14.01 ± 1.02 | 24.88 ± 6.89 | 63.27 ± 9.33 | 1.35 ± 3.12 |
| (g · kg⁻¹) | 4.0 | 5.08 ± 0.55 | 13.65 ± 1.21 | 23.52 ± 9.02 | 66.15 ± 10.56 | 0.85 ± 2.15 |

| | | Measurement indicators | | | | |
|---|---|---|---|---|---|---|
| Groups | | MO(%) | BA(%) | MCHC(Pg) | RDW(%) | PLT(K · μl⁻¹) |
| Control group | | 10.14 ± 4.15 | 0.19 ± 0.19 | 25.09 ± 3.92 | 16.28 ± 0.51 | 796.35 ± 214.25 |
| Sample | 1.0 | 10.96 ± 4.25 | 0.12 ± 0.15 | 26.75 ± 2.15 | 16.12 ± 0.93 | 811.42 ± 253.55 |
| group | 2.0 | 9.86 ± 3.62 | 0.36 ± 0.67 | 24.99 ± 2.97 | 17.61 ± 1.33 | 869.99 ± 205.12 |
| (g · kg⁻¹) | 4.0 | 10.25 ± 3.95 | 0.36 ± 0.32 | 26.16 ± 3.09 | 16.28 ± 1.26 | 763.12 ± 310.69 |

Note:
there was no significant difference in the body weight between the administration group and the normal control group after administration (P > 0.05)

Table 3 showed no significant difference in white blood cell count (WBC), percentage of neutrophils (NE %), percentage of lymphocytes (LY %), percentage of monocytes (MO %), percentage of eosinophils (EO %), basophils (BA %), red blood cell count (RBC), hemoglobin concentration (Hb), hematocrit (HCT %), mean corpuscular volume (MCV), mean corpuscular hemoglobin content (MCH), mean corpuscular hemoglobin concentration (MCHC), red blood cell distribution width (RDW %), platelet count (PLT), mean platelet volume (MPV) and other indicators between each dose group and the control group (P>0.05).

Blood biochemical indicators: Table 4 showed no significant difference in blood biochemical indicators such as glutamic pyruvic transaminase (GPT), glutamic oxalacetic transaminase (GOT), total protein (TP), albumin (ALB), blood urea nitrogen (BUN), creatinine (CRE), total cholesterol (CHO), triglyceride (TG), and glucose (GLU) between the mice in each dose group and the control group (P>0.05).

TABLE 4
Effects of the astragalus polysaccharide powder on blood biochemistry in Mice (x̅ ± s)

| | | Measurement indicators | | | | |
|---|---|---|---|---|---|---|
| | | TG | CHO | GOT | GPT | CRE |
| Groups | | mmol · L⁻¹ | mmol · L⁻¹ | U · L⁻¹ | U · L⁻¹ | mmol · L⁻¹ |
| Control group | | 139.93 ± 15.69 | 2.56 ± 0.65 | 89.35 ± 9.56 | 47.21 ± 11.25 | 77.26 ± 12.36 |
| Sample | 1.0 | 144.35 ± 8.69 | 2.95 ± 0.94 | 87.29 ± 8.63 | 46.29 ± 7.69 | 73.58 ± 9.18 |
| group | 2.0 | 151.25 ± 14.36 | 2.45 ± 0.62 | 86.33 ± 12.56 | 51.25 ± 9.89 | 77.25 ± 9.69 |
| (g · kg⁻¹) | 4.0 | 146.14 ± 9.66 | 2.76 ± 0.93 | 88.10 ± 11.15 | 50.11 ± 12.36 | 76.37 ± 12.95 |

Measurement indicators

| | | BUN | GLU | TP | ALB |
|---|---|---|---|---|---|
| Groups | | mol · L⁻¹ | mmol · L⁻¹ | g · L⁻¹ | g · L⁻¹ |
| Control group | | 7.19 ± 1.59 | 11.26 ± 2.95 | 60.74 ± 7.69 | 31.55 ± 5.06 |
| Sample | 1.0 | 7.69 ± 0.96 | 10.25 ± 2.36 | 62.14 ± 5.98 | 34.25 ± 4.45 |
| group | 2.0 | 8.02 ± 1.36 | 10.63 ± 2.88 | 58.45 ± 6.95 | 32.57 ± 3.98 |
| (g · kg⁻¹) | 4.0 | 7.99 ± 1.25 | 10.86 ± 1.96 | 61.25 ± 8.32 | 30.88 ± 4.82 |

Note:
there was no significant difference in the body weight between the administration group and the normal control group after administration (P > 0.05)

Pathological and histological examination of mice fed for 30 days: the size, shape, color, texture, etc. of organs such as the heart, liver, kidney, spleen, stomach, testicles, and ovaries in each group were comprehensively and carefully observed, and no abnormalities were found. There was no obvious degeneration or necrosis of the main organs, and their luster was normal.

The 30-day feeding experiment on mice showed that the feeding instant Astragalus polysaccharide immune-enhancing powder had high food safety.

### Experimental Example 3: Immune Protection Experiment of the Feeding Instant Astragalus Polysaccharide Immune-Enhancing Powder

Experimental method: after one week of adaptation, the animals were randomly divided into groups based on gender and body weight. Thirty-six young mice were randomly divided into three groups: negative control group, model control group, and Astragalus polysaccharide administration group, with 12 mice in each group, half male and half female. A suspension of the Astragalus polysaccharide powder was prepared using water as a solvent and administered to mice by gavage once a day for 30 consecutive days. The dosage was 200 mg kg⁻¹ d⁻¹, the animals were weighed once a week, and the dosage was adjusted according to changes in body weight. The mice in the model group were intraperitoneally injected with cyclophosphamide at a dose of 80 mg/kg/d for three consecutive days. The administration experiment was carried out. On the 9th, 10th, 11th, 19th, 20th and 21st days after the experiment, the mice were strengthened once again, that is, cyclophosphamide was intraperitoneally injected at a dose of 80 mg kg⁻¹ d⁻¹.

Experiment Results

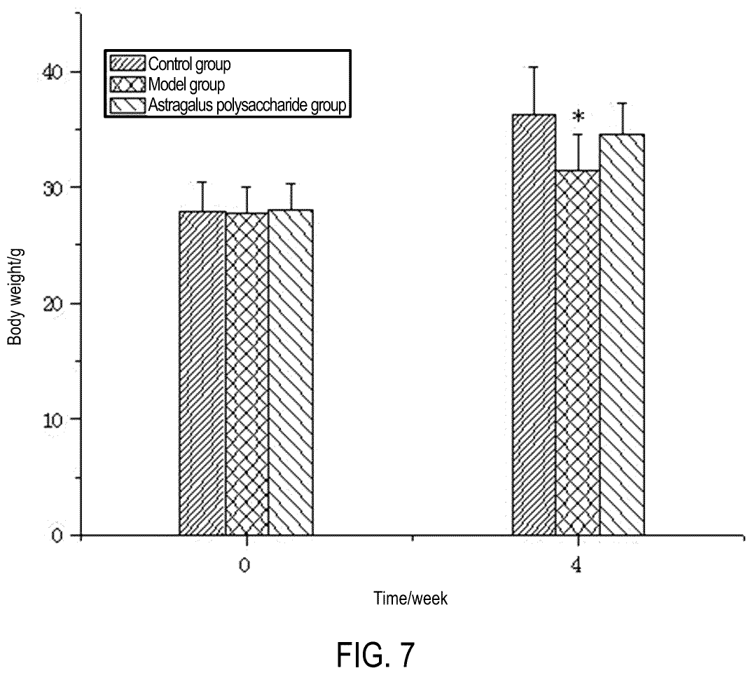

Effect of the Astragalus polysaccharide powder on the body weight of mice: the measurement results of the body weight of mice are shown in FIG. 7. FIG. 7 showed that after modeling with cyclophosphamide, the mice in the model group and other groups had normal food and water consumption, no abnormal changes in urination and defecation, and no abnormal deaths within the given modeling dose. However, the model group showed poor mental health, uneven hair color, and a significant decrease in weight gain, with a significant difference compared with the control group (P<0.05). The administration group can improve the weight gain of mice, and there was no significant difference compared with the negative control group (P>0.05).

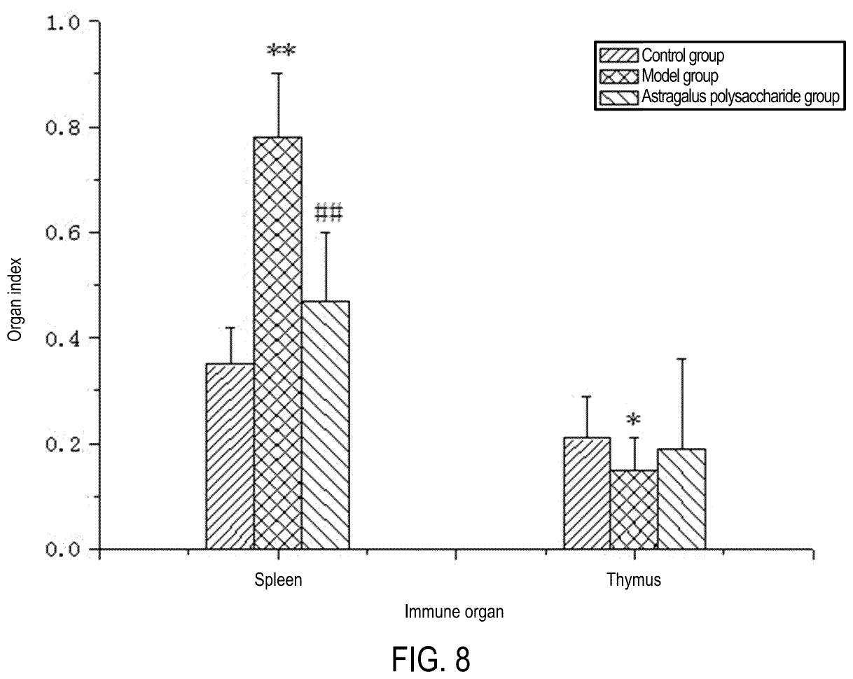

Effect of the Astragalus polysaccharide powder on the organ coefficients of immune organs in mice: the results of the organ coefficient measurement of immune organs in mice are shown in FIG. 8. FIG. 8 showed that compared with the control group, the thymus and spleen organ indices of mice in the model group changed, with a significant increase in the spleen index (P<0.01) and a significant decrease in the thymus index (P<0.05), indicating that the modeling method used in this experiment can effectively change the immune organ indices of mice. The Astragalus polysaccharide powder can reduce the spleen index and increase the thymus index, thereby repairing damaged immune organs.

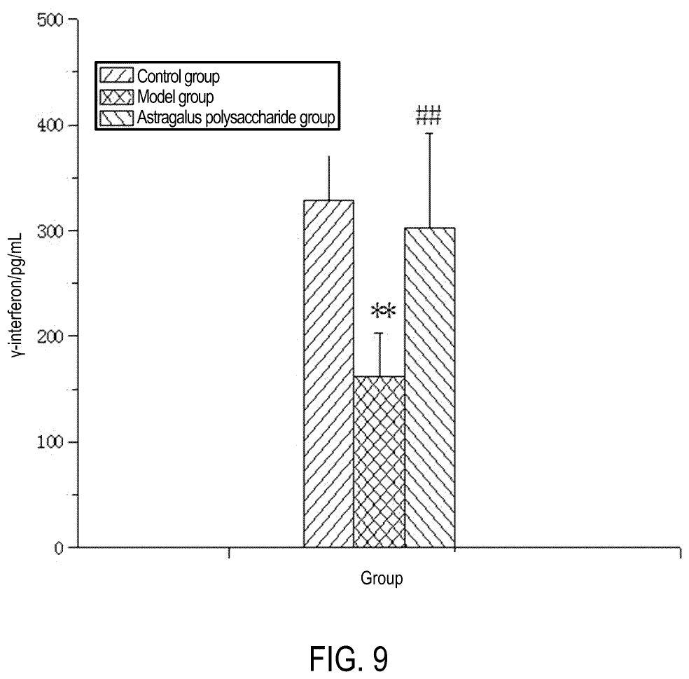

Effect of the Astragalus polysaccharide powder on γ-interferon in peripheral blood of mice: the ELISA method was used, and the operation method was carried out according to the instructions of the reagent kit. The detection results are shown in FIG. 9. γ-interferon is secreted by T cells and NK (natural killer) cells stimulated and activated by tumors, antigens, and mitotic factors. It has biological functions such as antiviral, immune-regulating, and anti-tumor properties. In addition, it can activate macrophages, enhance the cytotoxicity of natural killer cells, and stimulate the cytotoxicity of T cells. FIG. 9 showed that compared with the control group, the content of γ-interferon decreased significantly in mice of the model group (P<0.01), indicating that the immunosuppressive drug cyclophosphamide and modeling method used in this experiment can effectively reduce the γ-interferon levels, and inhibit the immune function of mice. Mice in the Astragalus polysaccharide sample group showed an extremely significant elevation in the level of γ-interferon which reached the range of the normal group (P>0.05), indicating that the Astragalus polysaccharide powder sample has the effect of improving γ-interferon levels in the body, enhancing immune function.

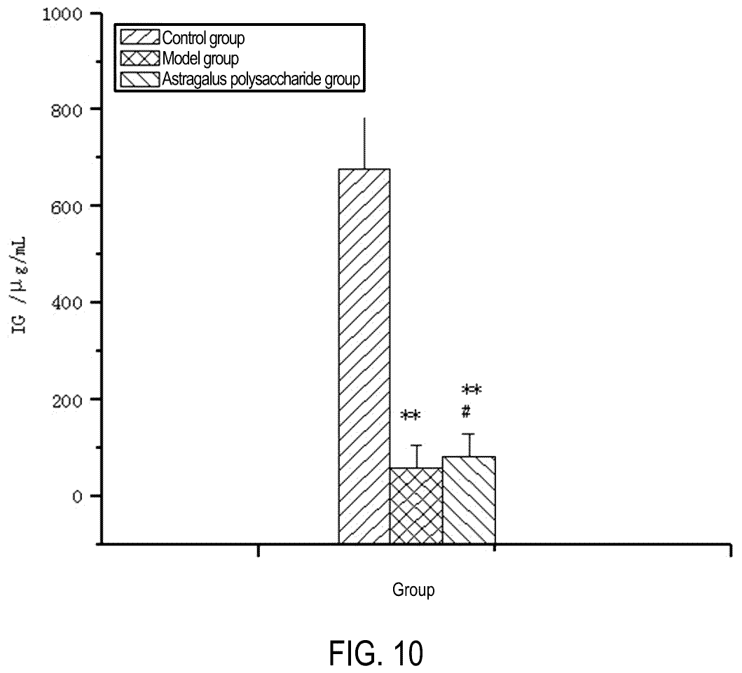

Effect of the Astragalus polysaccharide powder on IgG in peripheral blood of mice: determination of serum IgG in peripheral blood of mice: the ELISA method was used, and the operation method was carried out according to the instructions of the reagent kit. The detection results are shown in FIG. 10.

Immunoglobulin G (IgG) is synthesized in the spleen and lymph nodes. It has the highest content in serum (accounting for 75% of the amount of Ig), and has a long half-life in serum. It is mainly distributed in serum and interstitial fluid, and is the main component of anti-bacteria, anti-toxin and anti-virus antibodies, and also an important material basis in the process of anti-infection immunity of the body. Due to its highest content, IgG is the most important material basis for immune response. It has important immune effects and is the main force in the body's fight against infection. FIG. 9 showed that compared with the control group, the IgG content of the model group mice decreased significantly (P<0.01), further proving that the modeling method used in this experiment can effectively inhibit the immune function of mice. Compared with the model group, Astragalus polysaccharides significantly increased the IgG content (P<0.05).

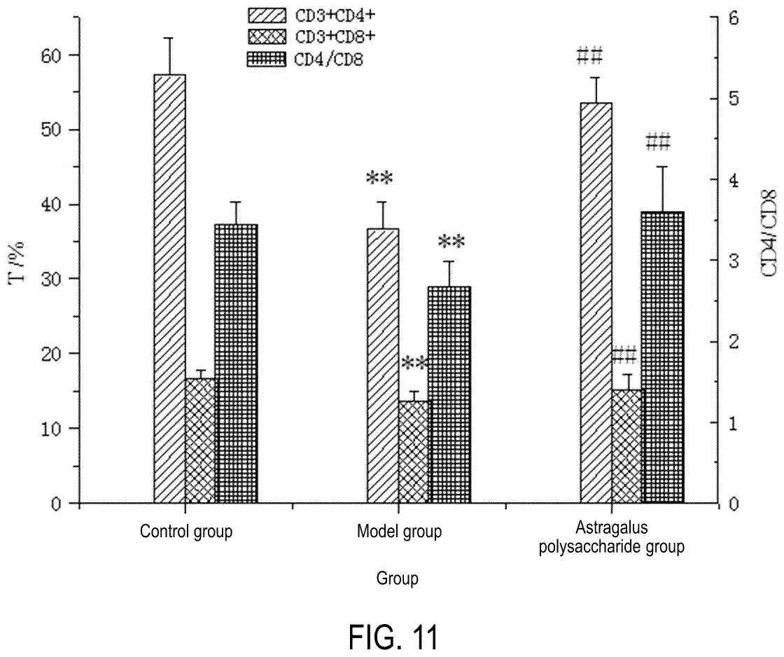

Effect of the Astragalus polysaccharide powder on T lymphocyte subpopulations in peripheral blood of mice: the lymphocyte subpopulations were analyzed using a flow cytometer, and the proportion of cells in the cross doors was statistically analyzed using BD CellQuest Pro data analysis software. The results of the effect of the Astragalus polysaccharide powder on T lymphocyte subpopulations in peripheral blood of mice are shown in FIG. 11. T lymphocytes play a very important role in cellular immunity and humoral immunity of the body, and have the functions of immune defense, immune regulation, immune surveillance, etc. Research data show that the balance of Th/Tc cells plays an important role in the occurrence and development of autoimmune diseases. CD4 is the surface marker of T helper/inducer cells (Th/Ti). CD4+ T cells have the functions of transforming helper T cells into effector cells, transforming B cells into plasma cells, and activating macrophages, and play the role of assisting and inducing cells and humoral immunity. CD8+ is the surface marker of T suppressor/killer cells (Ts/Tc). CD8+ T cells have cytotoxic effects, can inhibit the activation of T cells and the production of antibodies by B cells, and play a role in inhibiting cell and humoral immunity. Th cells are the central cells of the immune response in the body, and changes in the CD4/CD8 (Th/Tc) ratio reflect the immune function status of the body. An increase in the ratio indicates immune hyperfunction, while a decrease in the ratio indicates immune hypofunction. FIG. 10 showed that CD3+ CD4+, CD3+ CD8+ and CD4/CD8 in the model group decreased significantly (P<0.01), indicating that the immunosuppressive drug cyclophosphamide and modeling method used in this experiment can effectively inhibit the cellular immune function of mice. The Astragalus polysaccharide powder can significantly increase the number of CD3+ CD4+ (Th) cells in peripheral blood of immunosuppressed mice. In addition, the Astragalus polysaccharide powder can significantly increase the ratio of CD4/CD8 in peripheral blood of immunosuppressed mice (P<0.01), indicating that the Astragalus polysaccharide powder has a significant immune-enhancing effect.

What is claimed is:

1. A method for preparing a feeding instant Astragalus polysaccharide powder, the method comprising the following steps:
1) adding deionized water at 65° C. to a dreg of Astragalus membranaceus by water extraction and alcohol precipitation, and stirring until the dreg is completely dissolved to prepare a dreg solution;
2) after the dreg solution cools to room temperature, removing insoluble thermo-sensitive macromolecular proteins, polysaccharides and solid residues by centrifugation to obtain a clear supernatant;
3) adding twice mass of deionized water to the clear supernatant, stirring fully, and then performing a secondary ultrafiltration treatment by hollow fiber membranes with pore sizes of 100 KDa and 10 kDa in turn, and collecting 10-100 kDa polysaccharide components; and
4) concentrating the polysaccharide components with a molecular weight between 10-100 kDa collected in the step 3) to 40% of solid contents of the polysaccharide components under vacuum and low temperature, and spray drying to obtain the instant Astragalus polysaccharide powder.

2. The method for preparing the feeding instant Astragalus polysaccharide powder of claim 1, wherein in the step 1), an alcohol volume fraction of the dreg aqueous solution is controlled at 20%.

3. The method for preparing the feeding instant Astragalus polysaccharide powder of claim 1, wherein in the step 1), the dreg of Astragalus membranaceus by water extraction and alcohol precipitation is a semi-solid residue obtained by extracting traditional Chinese medicine Astragalus membranaceus pieces with hot water, removing a solid residue, and adding 3-4 times volume of anhydrous ethanol.

4. The method for preparing the feeding instant Astragalus polysaccharide powder of claim 1, wherein in the step 2), a speed of the centrifugation is 3500 revolutions per minute.

5. The method for preparing the feeding instant Astragalus polysaccharide powder of claim 1, wherein in the step 4), conditions of the spray drying are as follows: an atomizer frequency is 70 Hz, an inlet air temperature is controlled at 140° C., an outlet air temperature is controlled below 60° C., a drying tower interlayer is cooled by cold air, and a bottom of a drying tower is vibrated by a vibrating hammer every 5 minutes to prevent a wall and the bottom of the drying tower from powder deposition and liquefaction foaming.

6. An application of the feeding instant Astragalus polysaccharide powder prepared by the method according to claim 1 in a feed additive.

7. An application of the feeding instant Astragalus polysaccharide powder prepared by the method according to claim 2 in a feed additive.

8. An application of the feeding instant Astragalus polysaccharide powder prepared by the method according to claim 3 in a feed additive.

9. An application of the feeding instant Astragalus polysaccharide powder prepared by the method according to claim 4 in a feed additive.

10. An application of the feeding instant Astragalus polysaccharide powder prepared by the method according to claim 5 in a feed additive.
